(19) United States
(12) Patent Application Publication
(10) Pub. No.: US 2022/0296862 A1
(43) Pub. Date: Sep. 22, 2022

(54) Multi-Purpose Delivery Needle

(71) Applicant: Bard Access Systems, Inc., Salt Lake City, UT (US)

(72) Inventors: Kyle G. Thornley, Farmington, UT (US); Glade H. Howell, Draper, UT (US); Daniel B. Blanchard, Bountiful, UT (US); Joe Spataro, Cottonwood Heights, UT (US); Austin J. Mckinnon, Herriman, UT (US)

(21) Appl. No.: 17/700,152

(22) Filed: Mar. 21, 2022

Related U.S. Application Data
(60) Provisional application No. 63/164,191, filed on Mar. 22, 2021.

(51) Int. Cl. A61M 25/09; A61M 25/06

(52) U.S. Cl. CPC A61M 25/09041; A61M 25/065; A61M 25/0668

(57) ABSTRACT

A needle for accessing a vasculature of a patient is disclosed. The cannula of the needle includes a fluid lumen extending from the distal end to the proximal end and a guideway lumen extending proximally away from the distal end. The guideway lumen is configured for placement of a guidewire therein, and the guideway lumen includes a guideway slot extending along a length of the guideway lumen, where the guideway slot extends radially through a guideway-lumen wall and extends along the guideway lumen to the distal end. Also disclosed is a sheath covering the cannula and the sheath includes a separable portion through which guidewire may laterally pass. Also disclosed is a collar rotatably positionable between a guidewire secure position and a guidewire release position.

### PRIORITY

This application claims the benefit of priority to U.S. Provisional Application No. 63/164,191, filed Mar. 22, 2021, which is incorporated by reference in its entirety into this application.

### BACKGROUND

Various endovascular devices, including guidewires, catheters, vascular snares, or any other percutaneous instrument, may be inserted into the vasculature of a patient to detect and/or treat various health issues. The placement of the percutaneous instrument within the vasculature typically requires several steps. The steps may generally include accessing a blood vessel through the skin by piercing the skin and inserting the tip of the needle into the blood vessel. Typical practice may include establishing and maintaining a negative pressure within the needle during the needle insertion process to prevent introducing foreign matter into the patient including the blood vessel. As such a syringe is typically coupled with the needle to provide the negative pressure. After the tip of the needle is inserted into the blood vessel, the clinician may draw blood from the blood vessel into the syringe (commonly referred to as drawing flash) to confirm that the needle tip is within the blood vessel. Thereafter, the syringe is decoupled from the hub of the needle. The clinician may then insert the guidewire or other percutaneous instrument through the needle cannula and into the blood vessel. The percutaneous instrument is inserted into the blood vessel, the needle is removed from the instrument by sliding the needle off the proximal end of the instrument.

The typical vasculature access process described above presents a few complications. For example, care must be taken to ensure that the needle tip remains in the blood vessel until the guidewire has been inserted through the needle and into the blood vessel. It may be difficult for the clinician to maintain the tip of the needle within the blood vessel while decoupling the syringe from the needle especially in cases when the blood vessel is small. As such, the clinician may inadvertently displace the needle tip out of the blood vessel. Furthermore, once the syringe is decoupled from the needle, it is not possible for the clinician to reconfirm that the needle tip has remained within the blood vessel.

During the vasculature access process, the needle cannula serves two purposes. As described above, the cannula 1) defines fluid access to the blood vessel and 2) defines a guided pathway for the instrument. However, since the needle includes a single cannula, both purposes may not be satisfied at the same time. In other words, the cannula cannot be used as an instrument pathway when the needle is coupled to the syringe, and the needle cannot be used as a fluid access when the instrument is inserted through the cannula.

The current vasculature access process also complicates or limits the design of the percutaneous instrument. For example, the instrument must accommodate decoupling from the needle from the instrument via displacement of the proximal end of the instrument through the cannula.

Disclosed herein are new devices, systems, and methods for enhancing the safety and practicality of accessing the vasculature of the patient and placing a percutaneous instrument within the vasculature of the patient.

### SUMMARY

Disclosed herein is needle for accessing a vasculature of a patient. The needle includes a sharp tip at a distal end, a connection hub at a proximal end, where the hub is configured for connection to a syringe. The needle further includes a cannula extending from the distal end to the proximal end. The cannula includes a fluid lumen extending from the distal end to the proximal end and a guideway lumen extending proximally away from the distal end. The guideway lumen is configured for placement of a guidewire therein, and the guideway lumen includes a guideway slot extending along a length of the guideway lumen, where the guideway slot extends radially through a guideway-lumen wall and extends along the guideway lumen to the distal end. A width of the guideway slot may be substantially equal to or less than a diameter of the guideway lumen.

The needle may further include a sheath disposed over the cannula along a length of the cannula extending away from the distal end, where the sheath is configured to retain the guidewire within the guideway lumen. The sheath may include a sheath wall having a separable portion extending from a distal end of the sheath to a proximal end of the sheath. The sheath may be rotatable about the cannula between a first angular position and a second angular position, where the separable portion is disposed adjacent the guideway slot in the first position, and the separable portion is disposed away from the guideway slot in the second position. In some embodiments, the separable portion is configured for passage of the guidewire therethrough and in some embodiments, the separable portion is configured for passage of the cannula therethrough. The separable portion may include a slit.

In some embodiments, the guideway-lumen wall includes a deflectable portion extending at least partially across the guideway slot. The deflectable portion is configured to retain of the guidewire within the guideway slot when the deflectable portion is in a non-deflected state, and the deflectable portion is configured to allow passage of the guidewire out of the guideway slot when the deflectable portion is in a deflected state.

In some embodiments, the fluid lumen includes a fluid-lumen slot extending radially through a fluid-lumen wall, where the fluid-lumen slot extends away from the distal end along a length of the cannula, and a width of the fluid-lumen slot is substantially equal to or less than a diameter of the guideway lumen. The guideway-lumen wall extends across the fluid-lumen slot to define a portion of the fluid-lumen wall. The guideway-lumen wall and a fluid-lumen wall may be formed of different materials and the guideway-lumen wall may be more flexible than the fluid-lumen wall. In some embodiments, the fluid lumen comprises a non-circular cross section.

In some embodiments, the guideway slot includes a first slot portion having a first slot width and a second slot portion having a second slot width, where the second slot width is greater than the first slot width. The first slot portion may be disposed distal the second slot portion and the first slot portion may extend distally to the distal end of the needle. The first slot width is less than a diameter of the guidewire, and the second slot width may be greater than the diameter of the guidewire.

In some embodiments, the cannula comprises a non-circular cross section, and in some embodiments, the cannula has an oval cross section or a triangular cross section.

In some embodiments, the needle includes a collar coupled to the cannula, where the cannula is disposed within an opening of the collar, the opening extends from a distal end of the collar to a proximal end of the collar, and the collar prevents lateral displacement of the guidewire out of the guidewire lumen. The collar may include a collar slot extending radially outward from the opening to an outside surface of the collar, where the collar slot extends from the distal end of the collar to the proximal end of the collar, and the collar slot is configured for lateral passage of the guidewire therethrough.

The collar may be rotatably positionable on the cannula to a guidewire release position where the collar slot is in alignment with the guideway slot. When the collar is in the release position, lateral removal of the guidewire from the guideway lumen is allowed, and when the collar is rotated away from the release position, lateral removal of the guidewire from the guideway lumen is prevented. The collar may also be rotatably positionable on the cannula to a guidewire secure position, so that when the collar is in the guidewire secure position, longitudinal displacement of the guidewire within the guideway lumen is prevented, and when the collar is rotated away from the guidewire secure position, longitudinal displacement of the guidewire within the guideway lumen is allowed. In some embodiments, when the collar is in the guidewire secure position, rotation of the guidewire with respect to the needle is prevented, and when the collar is rotated away from the guidewire secure position, rotation of the guidewire with respect to the needle is allowed. The collar may be positioned on the cannula so that the second slot portion is disposed between the distal end of the collar and the proximal end of the collar. The collar may also be coupled to the sheath to define co-rotation therewith.

Also disclosed herein is a system for accessing a vasculature of a patient including a needle and a guidewire. The needle includes a sharp tip at a distal end and a connection hub at a proximal end, where the hub is configured for connection to a syringe. The needle further includes a cannula extending from the distal end to the proximal end. The cannula includes a fluid lumen extending from the distal end to the proximal end and a guideway lumen extending proximally away from the distal end. The guideway lumen is configured for placement of a guidewire therein, and the guideway lumen comprises a guideway slot extending along a length of the guideway lumen. The guidewire is disposed within the guideway lumen. The system may further include a syringe coupled to the needle.

In some embodiments of the system, the needle further includes a sheath disposed over the cannula along a length of the cannula extending away from the distal end, where the sheath is configured to retain the guidewire within the guideway lumen. The sheath includes a sheath wall having a separable portion extending from a distal end of the sheath to a proximal end of the sheath, and the separable portion is configured for passage of the guidewire therethrough. The sheath may be rotatable about the cannula between a first angular position and a second angular position, where the separable portion is disposed adjacent the guideway slot in the first position, and the separable portion is disposed away from the guideway slot in the second position.

In some embodiments of the system, the guideway-lumen wall comprises a deflectable portion extending at least partially across the guideway slot, where the deflectable portion is configured to retain the guidewire within the guideway slot when the deflectable portion is in a non-deflected state, and the deflectable portion is configured to allow passage of the guidewire out of the guideway slot when the deflectable portion is in a deflected state.

The guideway slot may include a first slot portion having a first slot width and a second slot portion having second slot width, where the second slot width is greater than the first slot width. The first slot width may be less than a diameter of the guidewire, and the second slot width may be greater than the diameter of the guidewire.

In some embodiments of the system, the guidewire includes a first diameter portion having a first diameter and a second diameter portion having a second diameter, where the first diameter is greater than the first slot width, and the second diameter is less than the first slot width.

In some embodiments of the system, the needle further includes a collar coupled to the cannula, the cannula is disposed within an opening of the collar, and the collar prevents lateral displacement of guidewire out of the guideway lumen. The opening extends from a distal end of the collar to a proximal end of the collar. The collar includes a collar slot extending radially outward from the opening to an outside surface of the collar, and the collar slot extends from the distal end of the collar to the proximal end of the collar. The collar slot is configured for lateral passage of the guidewire therethrough.

In some embodiments of the system, the collar is rotatably positionable on the cannula to a guidewire release position where the collar slot is in alignment with the guideway slot, so that when the collar is in the release position, lateral removal of the guidewire from the guideway lumen is allowed, and when the collar is rotated away from the release position, lateral removal of the guidewire from the guideway lumen is prevented.

The collar may also be rotatably positionable on the cannula to a guidewire secure position, so that when the collar is in the guidewire secure position, longitudinal displacement of the guidewire within the guideway lumen is prevented, and when the collar is rotated away from the guidewire secure position, longitudinal displacement of the guidewire within the guideway lumen is allowed. When the collar is in the guidewire secure position, rotation of the guidewire with respect to the needle may also be prevented, and when the collar is rotated away from the guidewire secure position, rotation of the guidewire with respect to the needle may be allowed.

Also disclosed herein is a method of placing a guidewire within a vasculature of a patient. The method includes the steps of inserting a needle through the skin of the patient so that a tip of the needle is disposed within a blood vessel, drawing blood from the blood vessel through a fluid lumen of the needle into a syringe coupled to the needle, advancing a guidewire through a guideway lumen of the needle along the blood vessel, and laterally separating the guidewire from the needle through a slot extending along the guideway lumen. The separating step may be performed while the syringe is coupled to the needle.

The method may further include a step of placing the guidewire in the guideway lumen so that a distal tip of the guidewire is disposed adjacent the tip of the needle and the placing step may be performed prior to the inserting step.

The method may further include a step of rotating a collar of the needle to a guidewire secure position prior to the inserting step to prevent longitudinal displacement of the guidewire within the guideway lumen during to the inserting step. The method may further include rotating the collar away from the secure position prior to the advancing step.

In some embodiments of the method, the separating step includes laterally displacing the guidewire through a separable portion of a sheath disposed over the cannula. In other embodiments, the separating step includes longitudinally displacing a reduced diameter portion of the guidewire through the slot.

These and other features of the concepts provided herein will become more apparent to those of skill in the art in view of the accompanying drawings and following description, which describe particular embodiments of such concepts in greater detail.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a vasculature access system, in accordance with some embodiments.

FIG. 1B is perspective detail view of a distal portion of the needle of FIG. 1A, in accordance with some embodiments.

FIG. 1C is perspective detail view similar to FIG. 1B further showing a sheath, in accordance with some embodiments.

FIG. 1D is perspective detail view similar to FIG. 1C further showing a separable portion of the sheath, in accordance with some embodiments.

FIG. 2A is perspective detail view of a distal portion of another embodiment of a needle, in accordance with some embodiments.

FIG. 2B is perspective detail view of a distal portion of another embodiment of the needle of FIG. 2A having a flexible wall portion, in accordance with some embodiments.

FIG. 2C is perspective detail view of a distal portion of another embodiment of the needle of FIG. 2A having a sheath, in accordance with some embodiments.

FIG. 3 is perspective detail view of a distal portion of another embodiment of a needle having a guideway lumen disposed within a fluid lumen, in accordance with some embodiments.

FIG. 4 is perspective detail view of a distal portion of another embodiment of a needle having a septum defining a fluid lumen and a guideway lumen within a cannula wall, in accordance with some embodiments.

FIG. 5 is perspective detail view of a distal portion of another embodiment of a needle having a guideway lumen with a flexible wall, in accordance with some embodiments.

FIG. 6A is perspective detail view of a distal portion of another embodiment of a needle having an oval shape, in accordance with some embodiments.

FIG. 6B is an end view of the needle of FIG. 6A, in accordance with some embodiments.

FIG. 7A is perspective detail view of a distal portion of another embodiment of a needle having a triangular shape, in accordance with some embodiments.

FIG. 7B is an end view of the needle of FIG. 7A, in accordance with some embodiments.

FIG. 8A is perspective detail view of a proximal portion of another embodiment of a needle having a slot with an expanded section, in accordance with some embodiments.

FIG. 8B is perspective detail view of the proximal portion of the needle of FIG. 8A illustrating a guidewire disposed within the slot, in accordance with some embodiments.

FIG. 9A is perspective view of a proximal portion of another embodiment of a needle having a rotatable collar coupled to the needle, in accordance with some embodiments.

FIG. 9B is back side perspective view of a proximal portion of the needle of FIG. 9A, in accordance with some embodiments.

FIG. 9C is a front view of a proximal portion of the needle of FIG. 9A with the collar rotated to a guidewire secure position, in accordance with some embodiments.

FIG. 9D is a cross-sectional view of a proximal portion of the needle of FIG. 9A cut along sectioning lines 9D-9D of FIG. 9C, in accordance with some embodiments.

FIG. 9E is a front view of a proximal portion of the needle of FIG. 9A with the collar rotated to a release position, in accordance with some embodiments.

FIG. 9F is a cross-sectional view of a proximal portion of the needle of FIG. 9A cut along sectioning lines 9F-9F of FIG. 9E, in accordance with some embodiments.

FIG. 9G is a perspective view of the needle of FIG. 9A including a sheath, in accordance with some embodiments.

FIG. 10A illustrates another embodiment of a vasculature access system, in accordance with some embodiments.

FIG. 10B is perspective detail view of a distal portion of the needle of FIG. 10A, in accordance with some embodiments.

FIG. 10C is a perspective view of a proximal portion of the needle of FIG. 10A showing a guidewire inserted through an expanded portion of a slot, in accordance with some embodiments.

FIG. 10D is a perspective view of a proximal portion of a system of FIG. 10A showing a guidewire having a reduced diameter portion, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The directional terms “proximal” and “distal” are used herein to refer to opposite locations on a medical device. The proximal end of the device is defined as the end of the device closest to the end-user and further from the patient when the device is in use by the end-user. The distal end is the end opposite the proximal end, along the longitudinal direction of the device, or the end furthest from the end-user and more near the patient.

The phrases “connected to” and “coupled to” refer to any form of interaction between two or more entities, including mechanical and fluid interaction. Two components may be connected or coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

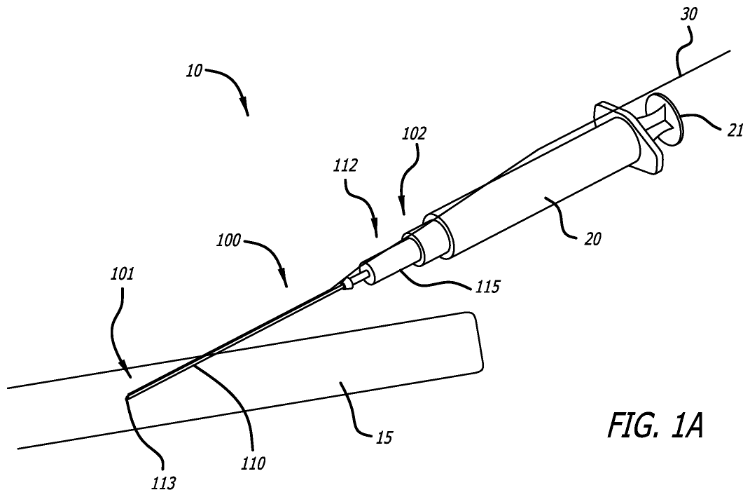

FIG. 1A illustrates a vasculature access system 10 including a needle 100, a syringe 20, and a guidewire 30. The needle 100 may be used to provide access for the guidewire 30 within a blood vessel 15. The needle 100 is coupled to the syringe 20 via a hub 115 disposed at a proximal end 102 of the needle 100, and the needle 100 includes a sharp tip 113 at a distal end 101. The needle 100 includes a cannula 110 extending from a proximal end 112 of the cannula adjacent the hub 115 to the tip 113. The cannula 110 may be formed of any rigid material such as stainless steel or a rigid plastic. As described further below, the needle 100 includes a fluid lumen defining fluid communication between the syringe 20 and the blood vessel 15.

In use, a clinician inserts the tip 113 of the needle 100 through the skin and into the blood vessel 15. In some instances, the clinician may withdraw the plunger 21 of the syringe 20 to draw blood from the blood vessel 15 for a visual “blood flash” confirmation that the tip 113 is within the blood vessel 15. The blood flash may be visualized within the syringe 20, along the cannula 110 when the cannula 110 is translucent/transparent, or in some other chamber. In other instances, the blood flash may include blood dripping from the needle hub 115 when the syringe 20 is decoupled from the hub 115. After confirmation that the tip 113 is within the blood vessel 15, the clinician may displace the guidewire distally into the blood vessel.

FIGS. 1B-1D illustrate is perspective detail views of a distal portion of the needle 100 showing additional details, components, and features of the needle 100. As shown in FIG. 1B, the cannula 110 includes a fluid lumen 120 having an open end adjacent the tip 113. The fluid lumen 120 extends from the tip 113 at the distal end 101 to the hub 115 at the proximal end 102 defining a fluid pathway along an entire length of the needle 100. The hub 115 facilitates physical attachment of the needle 100 to the syringe 20 and also establishes a fluid coupling of the fluid lumen 120 with the syringe 20.

The cannula 110 further incudes a guideway lumen 130 extending along a length the cannula 110 from the tip 113 to a proximal end 112 of the cannula 110. The guideway lumen 130 is sized to accommodate placement of the guidewire 30 therein. The cannula 110 includes a slot 135 extending longitudinally along the length of the guideway lumen 130. The slot 135 extends radially through a guideway lumen wall 131 defining a slot width 135A. The slot width 135A may be sized to accommodate lateral passage of the guidewire 30 into and out of the guideway lumen 130 through the slot 135. In some embodiments, the slot width 135A may be substantially equal to a diameter of the guideway lumen 130. In other embodiments, the slot width 135A may be less than a diameter of the guideway lumen 130. As shown, in use, the guidewire 30 may be positioned in the guideway lumen 130 so that a distal end 31 of the guidewire 30 is disposed adjacent the tip 113.

In the illustrated embodiment as shown in FIG. 1C, the needle 100 may include an elongate sheath 140 disposed about a circumference of the cannula 110. The sheath 140 may be sized to constrain the guidewire 30 within the guideway lumen 130. More specifically, the sheath 140 may prevent lateral displacement the guidewire 30 out of the guideway lumen 130. In some embodiments, the sheath 140 may be slidably coupled to the cannula 110 so that the sheath 140 may longitudinally slide and/or rotate with respect to the cannula 130. The sheath 140 may be sized and positioned on the cannula 130, so that the sharp tip 113 extends distally beyond a distal end of the sheath 140. Although not shown, the guideway lumen 130 extends proximally beyond a proximal end the sheath 140. The sheath 140 may be formed of a medical grade plastic material such as polypropylene, polyethylene, polytetrafluoroethylene (PTFE), or any other material suitable for forming a thin tubular sheath.

FIG. 1D is a perspective detail view similar to the FIG. 1C further showing a separable portion 141 of the sheath 140 which may extend along an entire length of the sheath 140. The separable portion 141 may be a slit or a longitudinal section of a weakened sheath structure such as a thin section or a perforation, for example. In use, the separable portion 141 may facilitate lateral removal of the guidewire 30 from the guideway lumen 130 upon a deliberate guidewire removal action by the clinician. The separable portion 141 may also facilitate retainment of the guidewire 30 within the guideway lumen 130 in the absence of a deliberate guidewire removal action by the clinician. In some embodiments, the deliberate guidewire removal action may include pulling on the guidewire with sufficient tension cause separation of the separable portion 141, thereby allowing lateral removal of the guidewire 30 from the guideway lumen 130. In some embodiments, the separable portion 141 may also facilitate lateral removal of the sheath 140 from the cannula 110.

In some embodiments, the separable portion 141 may be a slit having width greater than a diameter of the guidewire 30. In such an embodiment, the sheath 140 may retain the guidewire 30 within the guideway lumen 130 when the sheath 140 is rotatably positioned, so that the separable portion 141 is positioned away from the slot 135. In this embodiment, the deliberate guidewire removal action may include rotating the sheath 140 to align the separable portion 141 with the slot 135 thereby facilitating lateral removal of the guidewire 30 from the guideway lumen 130.

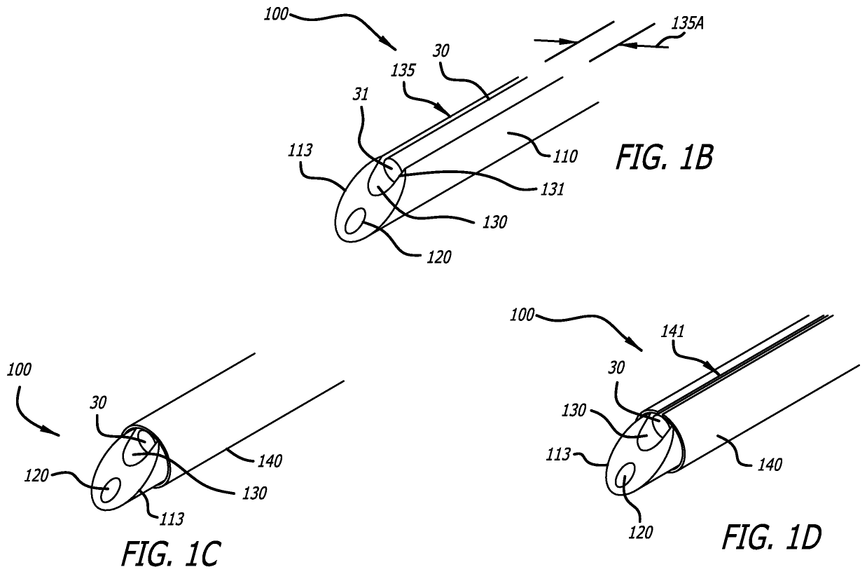

FIGS. 2A-2C illustrate embodiments of a needle 200 that can, in certain respects, resemble components of the needle 100 described in connection with FIGS. 1A-1D. It will be appreciated that all the illustrated embodiments may have analogous features. Accordingly, like features are designated with like reference numerals, with the leading digits incremented to “2.” For instance, the cannula is designated as “110” in FIGS. 1A-1D, and an analogous cannula is designated as “210” in FIGS. 2A-2C. Relevant disclosure set forth above regarding similarly identified features thus may not be repeated hereafter. Moreover, specific features of the needle 100 and related components shown in FIGS. 1A-1D may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the needle of FIGS. 2A-2C. Any suitable combination of the features, and variations of the same, described with respect to the needle 100 and components illustrated in FIGS. 1A-1D can be employed with the needle and components of FIGS. 2A-2C, and vice versa. This pattern of disclosure applies equally to further embodiments depicted in subsequent figures and described hereafter.

FIGS. 2A-2C are perspective detail views of a distal portion of the needle 200 showing details, components, and features of the needle 200. The needle 200 includes a cannula 210 having a cannula wall 211. As shown in FIGS. 1A and 1B, the cannula 210 includes a fluid lumen 220 having an open end adjacent the tip 213. The fluid lumen 220 extends from the tip 213 to the hub (not shown but see FIG. 1A) at the proximal end defining a fluid pathway along an entire length of the needle 200. The fluid lumen 220 is defined by a fluid lumen wall 221 forming a circumference of the fluid lumen 220. As such, the fluid lumen wall 221 may define a separate fluid conduit disposed within a cannula interior defined by the cannula wall 211.

The cannula wall 211 and the fluid lumen wall 221 combine with each other to define a guideway lumen 230 extending along a length the cannula 210 from the tip 213 to a proximal end of the cannula 210. The guideway lumen 230 is sized to accommodate placement of the guidewire 30 therein. The cannula 210 includes a longitudinal slot 235 extending along a length of the cannula 210 from the distal tip 213 to the hub. The slot 235 extends radially through the cannula wall 211 defining a slot width 235A. The slot width 235A may be sized to accommodate lateral passage of the guidewire 30 into and out of the guideway lumen 230. As shown, in use, the guidewire 30 may be positioned in the guideway lumen 230 so that a distal end 31 of the guidewire 30 is disposed adjacent the tip 213.

As shown in FIG. 2B, the cannula wall 211 may include one or more flexible extensions 211A. The flexible extensions 211A extend partially across the slot 235 to define reduced slot width 235B. The reduced slot width 235B may be less than a diameter of the guidewire 30, so that the flexible extensions 211A may facilitate retention of the guidewire 30 within the guideway lumen 230. The flexible extensions 211A may also facilitate lateral removal of the guidewire 30 from the guideway lumen 230 upon a deliberate guidewire removal action by the clinician.

In some embodiments, the cannula 210 can be made of metal, plastic, or other suitable material and the flexible extensions 211A may be extended portions of the cannula wall 211. In other embodiments, the flexible extensions 211A may be separate components attached to the cannula 210. In such embodiments, the flexible extensions 211A may be formed of any suitably flexible material, such as an elastomer including silicone, ethylene propylene diene monomer (EPDM), rubber, and the like or a flexible plastic.

In the illustrated embodiment as shown in FIG. 2C, the needle 100 may include an elongate sheath 240 disposed about a circumference of the cannula 210. The sheath 240 may be sized to constrain the guidewire 30 within the guideway lumen 230. More specifically, the sheath 140 may prevent lateral displacement the guidewire 30 out of the guideway lumen 230. In some embodiments, the sheath 240 may be slidably coupled to the cannula 210 so that the sheath 240 may longitudinally slide and/or rotate with respect to the cannula 210. The sheath 240 may be sized and positioned on the cannula 230, so that the sharp tip 213 extends distally beyond a distal end of the sheath 240. Although not shown, the guideway lumen 230 extends proximally beyond a proximal end the sheath 240.

The sheath 240 may include a separable portion 241 which may extend along an entire length of the sheath 240. The separable portion 241 may be a slit or a longitudinal section of a weakened structure such as a thin section or a perforation, for example. In use, the separable portion 241 may facilitate lateral removal of the guidewire 30 from the guideway lumen 230 upon deliberate action by the clinician. The separable portion 241 may also facilitate retainment of the guidewire 30 within the guideway lumen 230 in the absence of a deliberate action by the clinician. In some embodiments, the deliberate action may include pulling on the guidewire with sufficient tension cause separation of the separable portion 241, thereby allowing lateral removal of the guidewire 30 from the guideway lumen 230. In some embodiments, the separable portion 241 may also facilitate lateral removal of the sheath 240 from the cannula 210.

In some embodiments, the separable portion 241 may be a slit having width greater than a diameter of the guidewire 30. In such an embodiment, the sheath 240 may retain the guidewire 30 within the guideway lumen 230 when the sheath 240 is rotatably oriented, so that the separable portion 241 is positioned away from the slot 235. In this embodiment, the deliberate action may include rotating the sheath 240 to align the separable portion 241 with the slot 235 thereby facilitating lateral removal of the guidewire 30 from the guideway lumen 230.

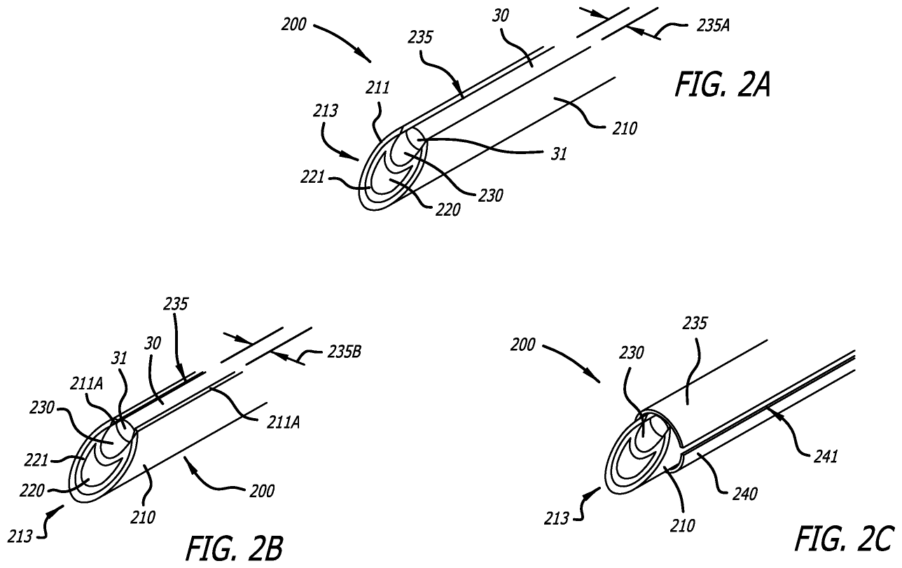

FIG. 3 is perspective detail view of a distal portion of a needle 300. The needle 300 includes a cannula 310 having a cannula wall 311, and the cannula wall 311 defines an interior of the cannula 310. A guideway lumen 330 including a guideway lumen wall 331 is disposed within the interior of the cannula 310, and the guideway lumen 330 extends along a length the cannula 310 from the tip 313 to a proximal end of the cannula 310. The cannula wall 311 and the guideway lumen wall 331 combine with each other to define the fluid lumen 320. The cannula wall 311 and the guideway lumen wall 331 are sealably coupled together to form a closed circumferential wall of the fluid lumen 320. The fluid lumen 320 extends from the tip 313 to the hub (not shown but see FIG. 1A) at the proximal end defining a fluid pathway along an entire length of the needle 300. As shown, the fluid lumen 320 extends at least partially around the guideway lumen 330.

The guideway lumen 330 is sized to accommodate placement of the guidewire 30 therein. The cannula wall 311 and the guideway lumen wall 331 include a longitudinal slot 335 extending along a length of the cannula 310 from the distal tip 313 to the hub. The slot 335 extends radially through the guideway lumen wall 331 and cannula wall 311 defining a slot width 335A. The slot width 335A may be sized to accommodate lateral passage of the guidewire 30 into and out of the guideway lumen 330. As shown, in use, the guidewire 30 may be positioned in the guideway lumen 330 so that a distal end 31 of the guidewire 30 is disposed adjacent the tip 313.

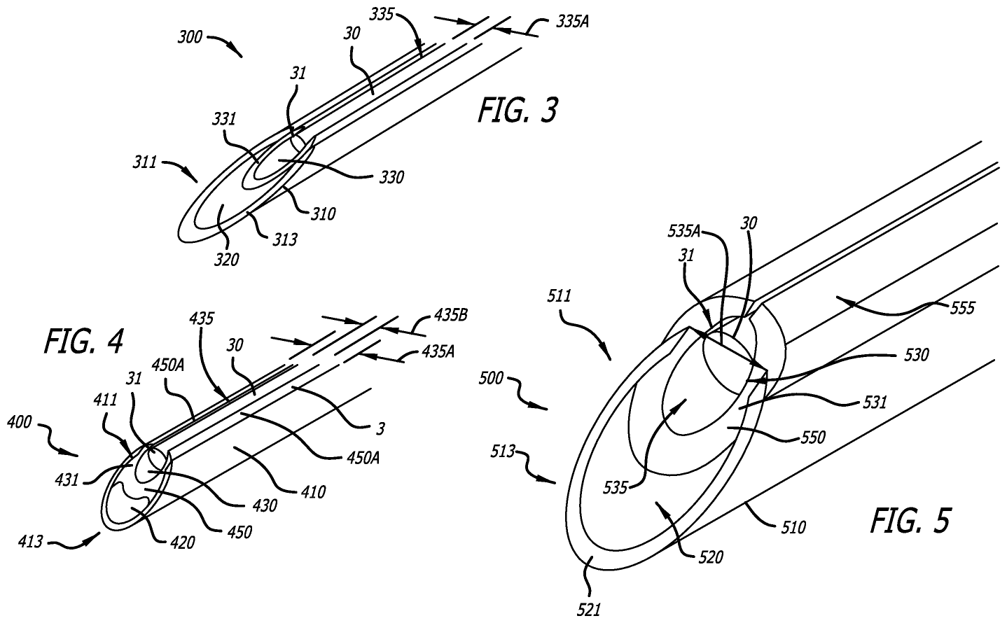

FIG. 4 is front perspective detail view of a distal portion of is front perspective detail view of a distal portion of a needle 400. The needle 400 includes a cannula 410 having a cannula wall 411, and the cannula wall 411 defines an interior of the cannula 410. Disposed within the interior is a septum 450 attached to the cannula wall 411. The septum 450 may be formed of a flexible material, such as an elastomeric material including silicone, ethylene propylene diene monomer (EPDM), rubber, or a flexible plastic. The septum 450 and the cannula wall 411 combine with each other to define a fluid lumen 420. More specifically, the septum 450 and the cannula wall 411 combine to form a circumferential fluid lumen wall 421. The fluid lumen 420 extends from the tip 411 to the hub (not shown but see FIG. 1A) at the proximal end defining a fluid pathway along an entire length of the needle 400.

The septum 450 forms a guideway lumen 430 including a guideway lumen wall 431. The guideway lumen 430 extends along a length the cannula 410 from the tip 413 to a proximal end of the cannula 410. The guideway lumen 430 is sized to accommodate placement of the guidewire 30 therein. The cannula wall 411 includes a longitudinal cannula slot 435 extending along a length of the cannula 410 from the distal tip 413 to the hub. The cannula slot 435 extends radially through the cannula wall 411 defining a slot width 435A. The slot width 435A may be sized to accommodate lateral passage of the guidewire 30 into and out of the guideway lumen 430. As shown, in use, the guidewire 30 may be positioned in the guideway lumen 430 so that a distal end 31 of the guidewire 30 is disposed adjacent the tip 413.

The septum 450 may extend within the cannula slot 435 to define a one or more flexible extensions 450A of the septum 450. The flexible extensions 450A may extend partially across the cannula slot 435 to define reduced slot width 435B. The reduced slot width 435B may be less than a diameter of the guidewire 30, so that the flexible extensions 450A may facilitate retention of the guidewire 30 within the guideway lumen 430. The flexible extensions 450A may also facilitate lateral removal of the guidewire 30 from the guideway lumen 430 upon a deliberate guidewire removal action by the clinician.

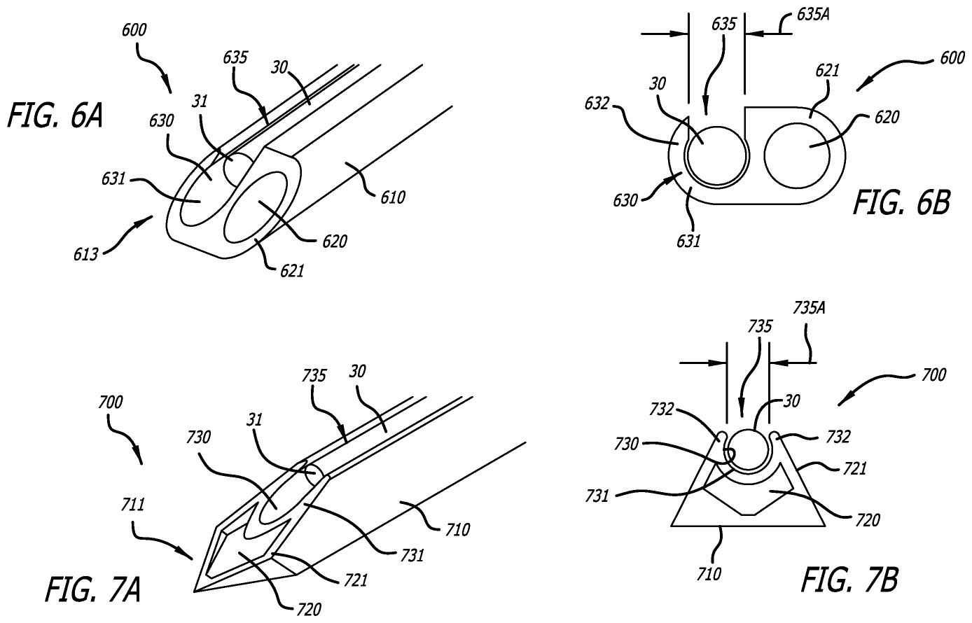

FIG. 5 is front perspective detail view of a distal portion of is front perspective detail view of a distal portion of a needle 500. The needle 500 includes a cannula 510 having a cannula wall 511, and the cannula wall 511 defines an interior of the cannula 510. Disposed at least partially within the interior is a septum 550 attached to an inside surface of the cannula wall 511. In some embodiments, a portion of the septum 550 may be attached to an outside surface of the cannula wall 511. The septum 550 may be formed of a flexible material, such as an elastomeric material including silicone, ethylene propylene diene monomer (EPDM), rubber, or a flexible plastic. The septum 550 and the cannula wall 511 combine with each other to define a fluid lumen 520. More specifically, the septum 550 and the cannula wall 511 combine to form a circumferential fluid-lumen wall 521. The fluid lumen 520 extends from the tip 513 to the hub (not shown but see FIG. 1A) at the proximal end defining a fluid pathway along an entire length of the needle 500.

The septum 550 forms a guideway lumen 530 including a guideway lumen wall 531. The guideway lumen 530 extends along a length the cannula 510 from the tip 511 to a proximal end of the cannula 510. The guideway lumen 530 is sized to accommodate placement of the guidewire 30 therein. The cannula wall 511 includes a longitudinal slot 535 extending along a length of the cannula 510 from the distal tip 511 to the hub. The cannula slot 535 extends radially through the cannula wall 511 defining a slot width 535A. The slot width 535A may be sized to accommodate lateral passage of the guidewire 30 into and out of the guideway lumen 530. As shown, in use, the guidewire 30 may be positioned in the guideway lumen 530 so that a distal end 31 of the guidewire 30 is disposed adjacent the tip 511.

The septum 550 may extend within the cannula slot 535. The septum 550 includes a slit 555 extending longitudinally along the septum 550. The slit 555 is positioned in alignment with the slot 535. The slit 555 extends radially through the guideway lumen wall 531 to define a one or more flexible extensions 550A of the septum 550. The flexible extensions 550A provide for retention of the guidewire 30 within the guideway lumen 530 in the absence of a deliberate guidewire removal action by the clinician. The flexible extensions 550A may also facilitate lateral removal of the guidewire 30 from the guideway lumen 530 upon a deliberate guidewire removal action by the clinician. In other words, the clinician my pull the guidewire 30 through the skit 555.

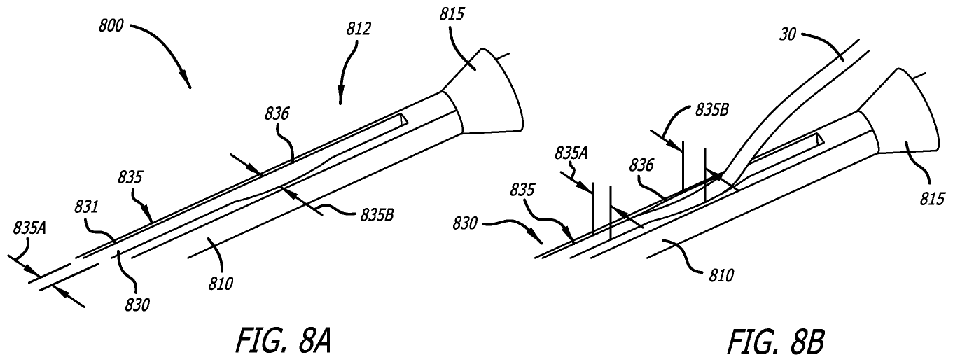

FIGS. 6A and 6B illustrate a needle 600. FIG. 6A is perspective detail view of a distal portion of the needle 600 and FIG. 6B is an end view of the distal portion of FIG. 6A. The needle 600 includes an oval shaped cannula 610 defining a fluid lumen 620 having fluid lumen wall 621 and a guideway lumen 630 having a guideway lumen wall 631. The fluid lumen 620 and guideway lumen 630 are positioned in a side by side relationship to define the oval shape. The fluid lumen 620 extends from the tip 613 to the hub (not shown but see FIG. 1A) at the proximal end defining a fluid pathway along an entire length of the needle 600.

The guideway lumen 630 extends along a length the cannula 610 from the tip 613 to a proximal end of the cannula 610. The guideway lumen 630 is sized to accommodate placement of the guidewire 30 therein. The guideway lumen wall 631 includes a longitudinal slot 635 extending along a length of the cannula 610 from the distal tip 613 to the hub. The cannula slot 635 extends radially through the guideway lumen wall 631 defining a slot width 635A. As shown, in use, the guidewire 30 may be positioned in the guideway lumen 630 so that a distal end 31 of the guidewire 30 is disposed adjacent the tip 613.

In the illustrated embodiment, the slot width 635A may be less than a diameter of the guidewire 30 so that the guidewire 30 is retained within the guideway lumen 630. The guideway-lumen wall 631 may include a deflectable portion 632 configured to deflect outward to increase the width 635A sufficiently to allow for lateral passage of the guidewire 30 out of the guideway lumen 630. In use, the deflectable portion 632 may facilitate retention of the guidewire 30 within the guideway lumen 630 in the absence of a deliberate guidewire removal action by the clinician. The deflectable portion 632 may also facilitate lateral removal of the guidewire 30 from the guideway lumen 630 upon a deliberate guidewire removal action by the clinician, such as pulling on the guidewire 30, for example.

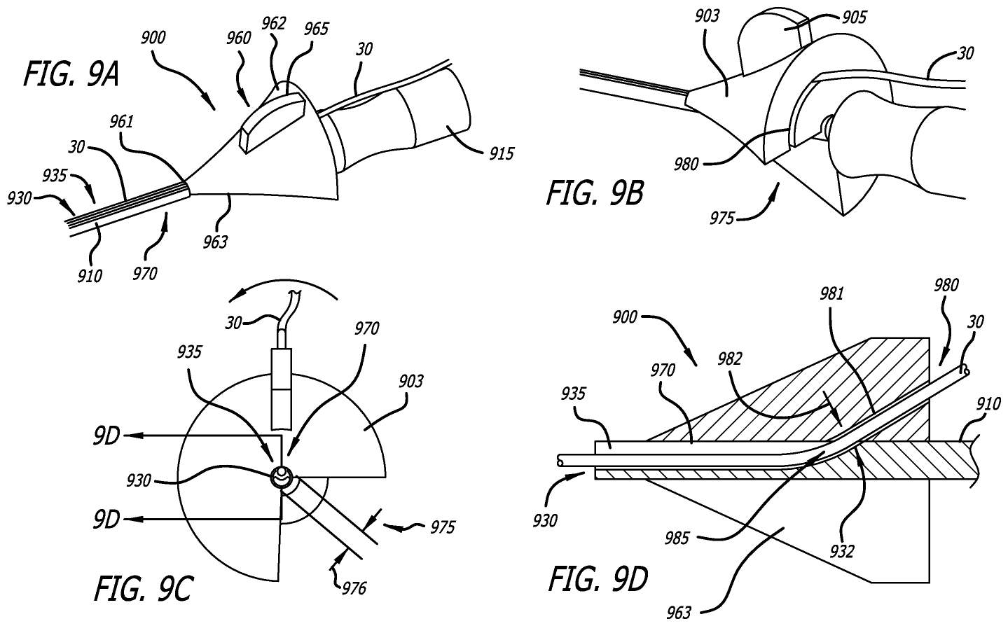

FIGS. 7A and 7B illustrate a needle 700. FIG. 7A is perspective detail view of a distal portion of the needle 700, and FIG. 7B is an end view of the distal portion of FIG. 7A. The needle 700 includes a triangular-shaped cannula 710 defining a fluid lumen 720 having fluid-lumen wall 721 and a guideway lumen 730 having a guideway-lumen wall 731. The fluid lumen 720 and guideway lumen 730 are positioned in a side by side relationship. The guideway lumen 730 may include circular cross-section and a cross section of fluid lumen 720 may be defined by the triangular shape of the cannula 710. The fluid lumen 720 extends from the tip 713 to the hub (not shown but see FIG. 1A) at the proximal end defining a fluid pathway along an entire length of the needle 700.

The guideway lumen 730 extends along a length the cannula 710 from the tip 713 to a proximal end of the cannula 710. The guideway lumen 730 is sized to accommodate placement of the guidewire 30 therein. The guideway lumen wall 731 includes a longitudinal slot 735 extending along a length of the cannula 710 from the distal tip 713 to the hub. The cannula slot 735 extends radially through the guideway lumen wall 731 defining a slot width 735A. As shown, in use, the guidewire 30 may be positioned in the guideway lumen 730 so that a distal end 31 of the guidewire 30 is disposed adjacent the tip 713.

In the illustrated embodiment, the slot width 735A may be less than a diameter of the guidewire 30 so that the guidewire 30 is retained within the guideway lumen 730. The guideway-lumen wall 731 may include one or more deflectable portions 732 configured to deflect outward to increase the width 735A sufficiently to allow for lateral passage of the guidewire 30 out of the guideway lumen 730. In use, the deflectable portions 732 may facilitate retention of the guidewire 30 within the guideway lumen 730 in the absence of a deliberate guidewire removal action by the clinician. The deflectable portions 732 may also facilitate lateral removal of the guidewire 30 from the guideway lumen 730 upon a deliberate guidewire removal action by the clinician, such as pulling on the guidewire, for example.

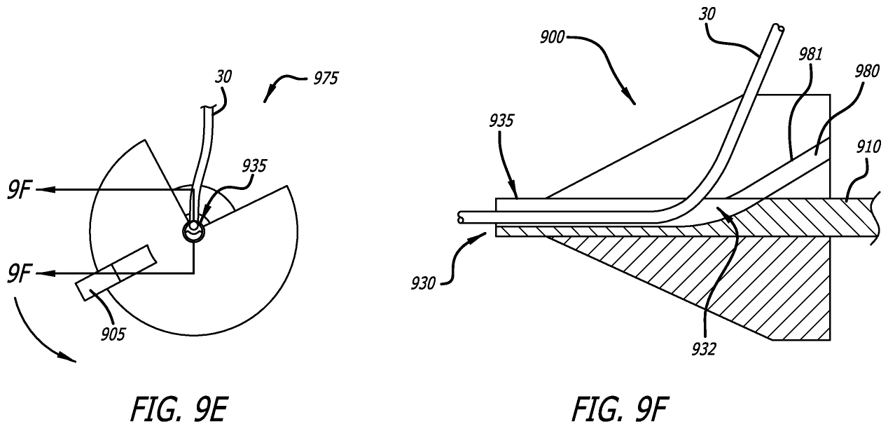

FIG. 8A is perspective detail view of a proximal portion of a needle 800. Shown is a cannula 810 extending distal away from a hub 815. The cannula 810 includes a guideway lumen 830 having a guideway lumen wall 831. The guideway lumen 830 extends along a length the cannula 810 from a distal end of the cannula 810 to a proximal end 812 of the cannula 810. The guideway lumen 830 is sized to accommodate placement of the guidewire 30 therein. The guideway lumen wall 831 includes a longitudinal slot 835 extending along a length of the cannula 810 from the distal end to the hub 815. The slot 835 extends radially through the guideway lumen wall 831 defining a slot width 835A. The slot 835 includes an expanded portion 836 defining an expanded slot width 835B.

FIG. 8B illustrates the needle 800 having a guidewire 30 disposed within the slot 835. In the illustrated embodiment, the slot width 835A is less than a diameter of the guidewire 30 so that the guidewire 30 is retained within the guideway lumen 830. Conversely, the expanded slot width 835B is greater than the diameter of the guidewire 30 so that the guidewire 30 may exit the guideway lumen 830 through the expanded portion 836. As shown, the expanded portion 836 may be positioned adjacent a proximal end 812 of the cannula 810.

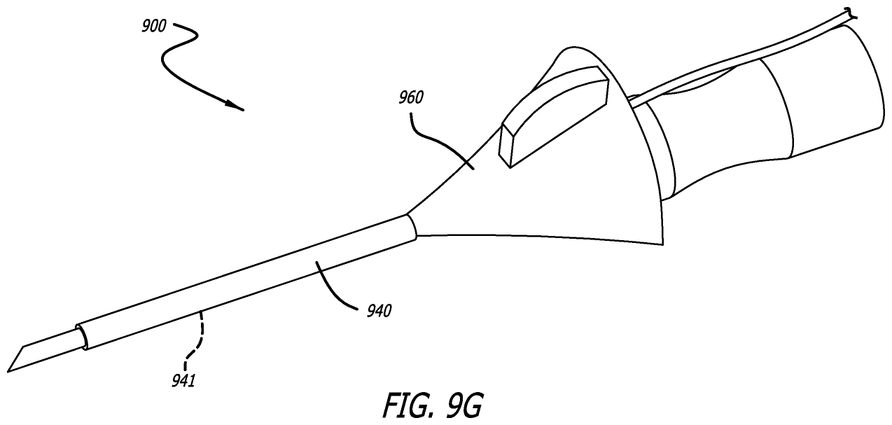

FIGS. 9A-9G illustrate another embodiment of a needle 900, in accordance with some embodiments. FIG. 9A is perspective view of a proximal portion of the needle 900 including a rotatable collar 960 coupled to the cannula 910. As shown, the collar 960 may be positioned adjacent the hub 915. The collar 960 defines a distal end 961, proximal end 962 and an outside surface 963. The collar 960 may include a handle 965 to facilitate rotation of the collar 960 by the clinician with respect to the hub 915. The collar 960 includes a cylindrical opening 970 (i.e., a hole) extending through the collar 960 from the distal end 961 to the proximal end 962, and the cannula 910 is inserted through the opening 970. The opening 970 is sized to defining a rotating and/or sliding fit with the cannula 910 and is also sized to prevent lateral displacement of the guidewire 30 out of the guideway lumen 930 via the guideway lumen slot 935.

The collar 960 is configured to rotate between a guidewire secure position as illustrated in FIGS. 9B-9D and a guidewire release position as illustrated in FIGS. 9E-9F. FIG. 9B is backside perspective view of proximal portion of the needle 900 showing the backside of the collar 960. The collar 960 includes a collar slot 975 extending radially outward from the opening 970 to the outside surface 963. The collar slot 975 also extends from the distal end 961 to the proximal end 962. As also shown, the guidewire 30 may be disposed within a passageway 980 of the collar 960.

FIG. 9C is a front view of a proximal portion of needle 900. A minimum width 976 of the collar slot 975 is sized small enough to prevent lateral displacement of the cannula 910 out of the opening 970 and large enough to allow lateral displacement of the guidewire 30 out of the opening 970.

FIG. 9D is a cross-sectional view of a proximal portion of the needle of FIG. 9A cut along sectioning lines 9D-9D of FIG. 9C illustrating a guidewire securing mechanism of the needle 900, in accordance with some embodiments. FIG. 9D illustrates the guidewire 30 disposed within the guideway lumen 930 along a distal portion of the collar 970. Also illustrated is the guidewire 30 exciting the guideway lumen 930 at a proximal end 932 of the guideway lumen 930. FIG. 9D further illustrates the guidewire 30 disposed within the passageway 980 along a proximal portion of the collar 970.

The guidewire securing mechanism may include a cam surface 981 defined by a portion of the passageway 980. The cam surface 981 defines a variable gap 985 between the cam surface 981 and the cannula 910 adjacent the proximal end 932 of the guideway lumen 930. The cam surface 981 may be configured so that the gap 985 is reduced when the collar 960 is rotated toward the guidewire secure position and increased when the collar 970 is rotated toward the guidewire release position. When the collar 960 is rotated to the guidewire secure position, the gap 985 may be sufficiently reduced to define a clamping force 982 on the guidewire 30. The clamping force 982 may be sufficient to prevent longitudinal displacement and/or rotation of the guidewire 30 with respect to the needle 900.

FIG. 9E is a front view of a proximal portion of the needle 900 and FIG. 9F is a cross-sectional view of a proximal portion of the needle 900 cut along sectioning lines 9F-9F of FIG. 9E with the collar 970 rotated to the guidewire release position. As shown in FIG. 9E, when the collar 970 is rotated to the guidewire release position, the collar slot 975 is aligned with the slot 935 of the guideway lumen 930. As such, the collar slot 975 allows for lateral removal of the guidewire 30 from the guideway lumen 930 via the slot 935. As shown in FIG. 9F, the guidewire 30 is removed from the passageway 980. With the collar 970 rotated to the guidewire release position, the clinician may laterally separate the needle 900 from the guidewire 30. In other words, the clinician may separate the guidewire 30 from the needle 900 without threading needle 900 off the proximal end of the guidewire 30.

In some embodiments, the guidewire 30 may be separated from the needle 900 by sliding the collar 970 distally off the distal end of the cannula 910 and then laterally passing the guidewire 30 through the slot 935. In some embodiments, the collar 970 may be formed of a flexible material or include one or more flexible portions (e.g., a hinge portion) so that the minimum width 976 of the slot 975 may widened via deformation of the collar 970. In such embodiments, the minimum width 976 of the slot 975 may be increased to facilitate lateral displacement of the cannula 910 through the slot 975.

FIG. 9G is perspective view of the needle 900 including a sheath 940. The sheath 940 is disposed along the cannula 910 to constrain the guidewire 30 within the guideway lumen 930. The sheath 940 may also comprise a separable portion 941 (hidden on the bottom side of the sheath 940 in FIG. 9G) extending along a length of the sheath 940. The sheath 940 may be coupled to collar 970 to define co-rotation therewith i.e., so that the sheath 940 is rotated when the clinician rotates the collar 960. The sheath 940 may be coupled with the collar 970 so that the separable portion 941 is in angular alignment with the collar slot 975. In use, the clinician may rotate the collar 960 to the guidewire release position and remove the guidewire 30 from the guideway lumen 930 by pulling the guidewire radially outward through the slot 935, collar slot 975, and the separable portion 941.

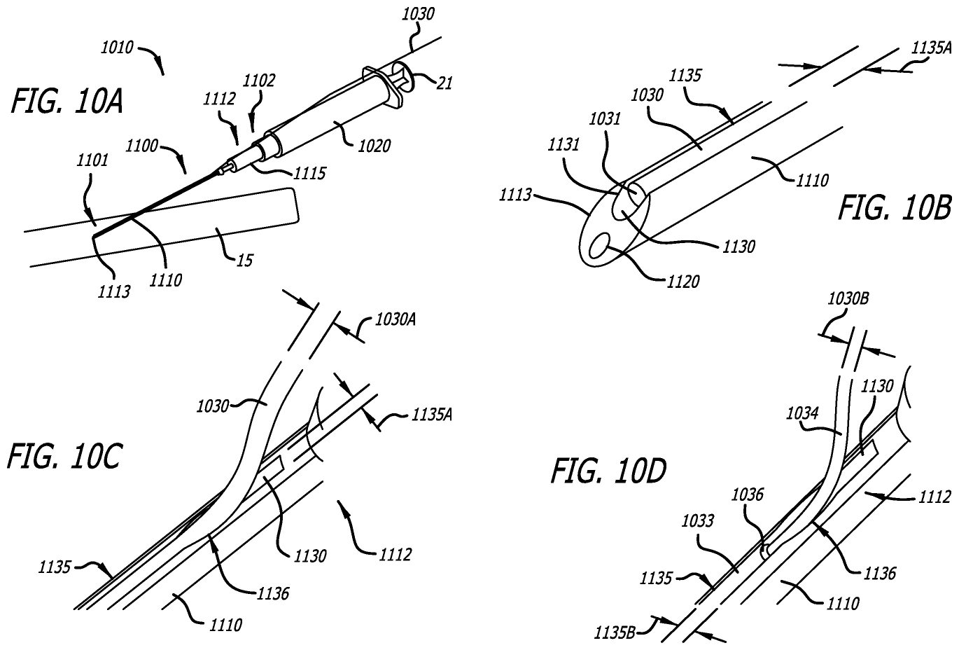

FIG. 10A illustrates another embodiment of a vasculature access system 1010 including a needle 1100, syringe 1020 and a guidewire 1030. The needle 1100 is coupled to the syringe 1020 via a hub 1115 disposed at a proximal end 1102 of the needle 1100 and includes a sharp tip 1113 at a distal end 1101. The needle 1100 includes a cannula 1110 extending from a proximal end 1112 of the cannula 1110 adjacent the hub 1115 to the tip 1113. The cannula 1110 may be formed of any rigid material such as stainless steel or a rigid plastic. The needle 1100 is used to provide access for a guidewire 1030 within a blood vessel 15. As described further below, the needle 1100 includes a fluid lumen defining fluid communication between the syringe 1020 and the blood vessel 15.

FIG. 10B is a perspective detail view of a distal portion of the needle 1100 showing additional details, components, and features of the needle 1100. As shown in FIG. 10B, the cannula 1110 includes a fluid lumen 1120 having an open end adjacent the tip 1113. The fluid lumen 1120 extends from the tip 1113 at the distal end 1101 to the hub 1115 at the proximal end 1102 defining a fluid pathway along an entire length of the needle 1100. The hub 1115 facilitates physical attachment of the needle 1100 to the syringe 1020 and also establishes a fluid coupling of the fluid lumen 1120 with the syringe 1020.

The cannula 1110 further incudes a guideway lumen 1130 extending along a length the cannula 1110 from the tip 1113 to a proximal end 1112 of the cannula 1110. The guideway lumen 1130 is sized to accommodate placement of the guidewire 1030 therein. The cannula 1110 includes a slot 1135 extending longitudinally along the length of the guideway lumen 1130. The slot 1135 extends radially through a guideway-lumen wall 1131 defining a slot width 1135A.

FIG. 10C is a perspective detail view of a proximal portion of the needle 1100 including a portion of the guidewire 1030 having a diameter 1030A. The slot width 1135A is sized to be less than the diameter 1030A of the guidewire 1030. FIG. 10C shows the guidewire 1030 having the diameter 1030A inserted into the guideway lumen 1030 through an expanded portion 1136 of the slot 1135. The expanded portion 1136 defines an expanded slot width 1135B (see FIG. 10D). The expanded slot width 1135B is sized to be larger than the diameter 1030A of the guidewire 1030.

FIG. 10D illustrates a general portion 1033 of the guidewire 1030 having the diameter 1030A and a reduced portion 1034 of the guidewire 1030 having a reduced diameter 1030B. The change of the diameter of guidewire 1030 from diameter 1030A to the reduced diameter 1030B occurs at the transition point 1036. The reduced diameter 1035B is sized to be less than the slot width 1135A. By way of summary, the general portion 1033 having the diameter 1030A may not laterally pass through the slot 1135 having a width 1135A but may pass through the expanded portion 1136 of the slot 1135 having a width 1135B. Similarly, the reduced portion 1034 of the guidewire 1030 having the reduced diameter 1030B may laterally pass through the slot 1135 having a width 1135A.

In use, the guidewire 30 may be inserted distally through the expanded portion 1136 into the guideway lumen 1130. The guidewire 30 may further be advanced along the guideway lumen 1130 and into the vasculature while general portion 1033 extends through the expanded portion 1136 of the slot 1135. The advancement may continue until the reduced portion 11134 of the guidewire 30 is positioned adjacent the proximal end 1112 of the cannula 1110. At this point the needle 1100 may be displaced proximally with respect the guidewire 30 so that the reduced portion 1134 is longitudinally displaced within the slot 1135. The proximal displacement of the needle 1100 may continue during which the reduced portion 1134 is longitudinally displaced along the slot 1135 until the reduced portion 1134 exits the distal end of the slot 1135 establishing separation of the needle 1100 from the guidewire 30.

A method of placing the guidewire 30 within a vasculature of a patient 50 may include the following steps or processes. The guidewire may be inserted into the guideway lumen of the needle so that the distal tip of the guidewire is disposed adjacent the distal tip of the needle. The needle may be inserted through the skin of the patient 50 so that the tip of the needle is disposed within a blood vessel. With the tip of the needle disposed within the blood vessel, blood may be drawn through the fluid lumen of the needle and into a syringe to confirm that the needle tip is within the blood vessel. The guidewire may be advanced through guideway lumen of the needle and along the blood vessel. The needle may be laterally separated from the guidewire while the syringe is coupled to the needle. The step of inserting the guidewire into the guideway lumen may be performed prior to the step of inserting the needle through the skin.

The collar may be rotated to secure the guidewire to the needle prior to the inserting step to prevent longitudinal displacement of the guidewire within the guideway lumen during insertion of the needle through the skin. The collar may be rotated away from the secure position so that the guidewire may be advanced through the needle.

The method may include using a needle having a sheath and the method may further include separating needle from the guidewire by laterally displacing the guidewire through a separable portion of the sheath. The guidewire may be laterally displaced through a slot of the collar when separating the needle from the guidewire.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A needle for accessing a vasculature of a patient, comprising:
a sharp tip at a distal end;
a connection hub at a proximal end, the hub configured for connection to a syringe;
a cannula extending from the distal end to the proximal end, the cannula comprising a fluid lumen extending from the distal end to the proximal end; and
a guideway lumen extending proximally away from the distal end, wherein the guideway lumen comprises a guideway slot extending along a length of the guideway lumen, and wherein the guideway lumen is configured for placement of a guidewire therein.

2. The needle according to claim 1, wherein the guideway slot extends radially through a guideway-lumen wall.

3. The needle according to claim 1, wherein the guideway slot extends along the guideway lumen to the distal end.

4. The needle according to claim 1, wherein a width of the guideway slot is substantially equal to or less than a diameter of the guideway lumen.

5. The needle according to claim 1, further comprising a sheath disposed over the cannula along a length of the cannula extending away from the distal end, the sheath configured to retain the guidewire within the guideway lumen.

6. The needle according to claim 5, wherein the sheath includes a sheath wall having a separable portion extending from a distal end of the sheath to a proximal end of the sheath.

7. The needle according to claim 6, wherein:
the sheath is rotatable about the cannula between a first angular position and a second angular position,
the separable portion is disposed adjacent the guideway slot in the first position, and
the separable portion is disposed away from the guideway slot in the second position.

8. The needle according to claim 5, wherein separable portion is configured for passage of the guidewire therethrough.

9. The needle according to claim 5, wherein separable portion is configured for passage of the cannula therethrough.

10. The needle according to claim 6, wherein the separable portion comprises a slit.

11. The needle according to claim 1, wherein:
the guideway-lumen wall comprises a deflectable portion extending at least partially across the guideway slot,
the deflectable portion is configured to retain of the guidewire within the guideway lumen when the deflectable portion is in a non-deflected state, and
the deflectable portion is configured to allow passage of the guidewire out of the guideway lumen when the deflectable portion is in a deflected state.

12. The needle according to claim 1, wherein:
the fluid lumen comprises a fluid-lumen slot extending radially through a fluid-lumen wall,
the fluid-lumen slot extends away from the distal end along a length of the cannula,
a width of the fluid-lumen slot is substantially equal to or less than a diameter of the guideway lumen, and
the guideway-lumen wall extends across the fluid-lumen slot to define a portion of the fluid-lumen wall.

13. The needle according to claim 1, wherein the guideway-lumen wall and a fluid-lumen wall are formed of different materials.

14. The needle according to claim 13, wherein the guideway-lumen wall is more flexible than the fluid-lumen wall.

15. The needle according to claim 1, wherein the fluid lumen comprises a non-circular cross section.

16. The needle according to claim 1, wherein the guideway slot comprises:
a first slot portion having a first slot width; and
a second slot portion having second slot width, wherein the second slot width is greater than the first slot width.

17. The needle according to claim 16, wherein the first slot portion is disposed distal the second slot portion.

18. The needle according to claim 16, wherein the first slot portion extends distally to the distal end of the needle.

19. The needle according to claim 16, wherein:
the first slot width is less than a diameter of the guidewire, and
the second slot width is greater than the diameter of the guidewire.

20. The needle according to claim 1, wherein the cannula comprises a non-circular cross section.

21. The needle according to claim 20, wherein the cannula comprises an oval cross section.

22. The needle according to claim 20, wherein the cannula comprises triangular cross section.

23. The needle according to claim 1, further comprising a collar coupled to the cannula, wherein:
the cannula is disposed within an opening of the collar,
the opening extends from a distal end of the collar to a proximal end of the collar, and
the collar prevents lateral displacement of the guidewire out of the guideway lumen.

24. The needle according to claim 23, wherein:
the collar includes a collar slot extending radially outward from the opening to an outside surface of the collar,
the collar slot extends from the distal end of the collar to the proximal end of the collar, and
the collar slot is configured for lateral passage of the guidewire therethrough.

25. The needle according to claim 24, wherein the collar is rotatably positionable on the cannula to a guidewire release position where the collar slot is in alignment with the guideway slot, such that:
when the collar is in the release position, lateral removal of the guidewire from the guideway lumen is allowed, and
when the collar is rotated away from the release position, lateral removal of the guidewire from the guideway lumen is prevented.

26. The needle according to claim 23, wherein the collar is rotatably positionable on the cannula to a guidewire secure position, such that:
when the collar is in the guidewire secure position, longitudinal displacement of the guidewire within the guideway lumen is prevented, and
when the collar is rotated away from the guidewire secure position, longitudinal displacement of the guidewire within the guideway lumen is allowed.

27. The needle according to claim 26, wherein:
when the collar is in the guidewire secure position, rotation of the guidewire with respect to the needle is prevented, and
when the collar is rotated away from the guidewire secure position, rotation of the guidewire with respect to the needle is allowed.

28. The needle according to claim 23, wherein the collar is positioned on the cannula so that the second slot portion is disposed between the distal end of the collar and the proximal end of the collar.

29. The needle according to claim 23, wherein the collar is coupled to the sheath to define co-rotation therewith.

30-50. (canceled)
